(19) United States
(12) Patent Application Publication
(10) Pub. No.: US 2022/0095931 A1
(43) Pub. Date: Mar. 31, 2022

(54) BREATH CLASSIFICATION SYSTEMS AND METHODS

(71) Applicant: Cardiac Pacemakers, Inc., St. Paul, MN (US)

(72) Inventors: Jeffrey E. Stahmann, Ramsey, MN (US); Viktoria A. Averina, Shoreview, MN (US)

(21) Appl. No.: 17/486,497

(22) Filed: Sep. 27, 2021

Related U.S. Application Data
(60) Provisional application No. 63/085,954, filed on Sep. 30, 2020.

(51) Int. Cl. A61B 5/0205; A61B 5/08; A61B 5/026; A61B 5/053; A61B 5/318; A61B 7/00; A61B 5/00

(52) U.S. Cl. CPC A61B 5/0205; A61B 5/0816; A61B 5/026; A61B 2562/0219; A61B 5/318; A61B 7/00; A61B 5/6847; A61B 5/053

(57) ABSTRACT

Systems and methods to determine a composite respiratory vibration of a patient are disclosed, including a signal receiver circuit configured to receive physiologic information cyclic with patient respiration and vibration information indicative of patient respiratory vibrations for a plurality of respiratory cycles of a patient, and an assessment circuit configured to identify a first set of respiratory cycles of the plurality of respiratory cycles having a duration within a threshold, align segments of the vibration information corresponding to the first set of respiratory cycles, the segments associated with a desired portion of the respiratory cycle using a feature of the respiratory cycle, and determine the composite respiratory vibration using the aligned segments.

### CLAIM OF PRIORITY

This application claims the benefit of priority of U.S. Provisional Patent Application Ser. No. 63/085,954, filed on Sep. 30, 2020, which is herein incorporated by reference in its entirety.

### TECHNICAL FIELD

This document relates generally to detecting patient respiration, and more particularly, but not by way of limitation, to systems and methods for patient breath classification.

### BACKGROUND

Normal patient respiration is automatic, and functions to provide sufficient oxygen (O₂) supply to the body and remove carbon dioxide (CO₂) to maintain a suitable acid-base status. Medical sensors or devices can detect or monitor respiration, such as to determine one or more respiratory parameters (e.g., respiratory rate, tidal volume, etc.), detect patient respiratory vibrations, such as patient respiratory sounds, or to determine periods of patient inspiration or expiration.

Ambulatory medical devices (AMDs) include implantable, subcutaneous, wearable, holdable, external, or one or more other type of medical devices having sensors configured to sense physiologic signals from a patient. Detected physiologic signals can be used to determine or monitor patient status or condition. Frequent patient monitoring, such as using one or more AMDs, can enable early detection of worsening patient condition or identification of patients or groups of patients having elevated risk of future adverse events, including hospitalization. Early detection of worsening patient condition can prevent or reduce patient hospitalization. Identifying and safely managing patient risk of worsening condition may reduce patient hospitalizations, the amount or severity of medical interventions, and overall healthcare costs.

### SUMMARY

Systems and methods to determine a composite respiratory vibration of a patient are disclosed, including a signal receiver circuit configured to receive physiologic information cyclic with patient respiration and vibration information indicative of patient respiratory vibrations for a plurality of respiratory cycles of a patient, and an assessment circuit configured to identify a first set of respiratory cycles of the plurality of respiratory cycles having a duration within a threshold, align segments of the vibration information corresponding to the first set of respiratory cycles, the segments associated with a desired portion of the respiratory cycle using a feature of the respiratory cycle, and determine the composite respiratory vibration using the aligned segments.

An example (e.g., “Example 1”) of subject matter (e.g., a system) may comprise a signal receiver circuit configured to receive physiologic information cyclic with patient respiration and vibration information indicative of patient respiratory vibrations for a plurality of respiratory cycles of a patient and an assessment circuit configured to identify a first set of respiratory cycles of the plurality of respiratory cycles having a duration within a threshold, align segments of the vibration information corresponding to the first set of respiratory cycles, the segments associated with a desired portion of the respiratory cycle using a feature of the respiratory cycle, and determine a composite respiratory vibration using the aligned segments.

In Example 2, the subject matter of Example 1 may optionally be configured to comprise an implantable housing comprising a vibration sensor configured to sense the vibration information indicative of patient respiratory vibrations.

In Example 3, the subject matter of any one or more of Examples 1-2 may optionally be configured such that the assessment circuit is configured to determine the composite respiratory vibration to improve a signal-to-noise ratio (SNR) of the vibration information.

In Example 4, the subject matter of any one or more of Examples 1-3 may optionally be configured such that the physiologic information and the vibration information comprise different types of information and the physiologic information comprises at least of electrocardiogram information of the patient, accelerometer information of the patient, impedance information of the patient, acoustic information of the patient, or blood flow information of the patient.

In Example 5, the subject matter of any one or more of Examples 1˜4 may optionally be configured such that the assessment circuit is configured to determine a change in patient status, to detect a physiological condition of the patient, or to determine a patient therapy parameter using the determined composite respiratory vibration.

In Example 6, the subject matter of any one or more of Examples 1-5 may optionally be configured such that the first set of respiratory cycles of the patient comprises at least 3 respiratory cycles.

In Example 7, the subject matter of any one or more of Examples 1-6 may optionally be configured such that the threshold comprises a range within N % of the duration a first respiratory cycle or of the inspiration/expiration (I/E) ratio of the first respiratory cycle.

In Example 8, the subject matter of any one or more of Examples 1-7 may optionally be configured such that the feature of the respiratory cycle comprises a transition between inspiration and expiration of the patient.

In Example 9, the subject matter of any one or more of Examples 1-8 may optionally be configured such that the composite respiratory vibration comprises an average of the aligned vibration information.

In Example 10, the subject matter of any one or more of Examples 1-9 may optionally be configured such that the segments of the vibration information associated with the desired portion of the respiratory cycle comprise segments associated with two or more of: a wheeze segment of the respiratory cycle; a stridor segment of the respiratory cycle; a squawk segment of the respiratory cycle; a rhonchus segment of the respiratory cycle; a snore segment of the respiratory cycle; a fine crackle segment of the respiratory cycle; a course crackle segment of the respiratory cycle; a crackle segment of the respiratory cycle; and a pleural friction rub segment of the respiratory cycle, and the assessment circuit is configured to determine composite respiratory vibrations for each of the two or more segments.

In Example 11, the subject matter of any one or more of Examples 1-10 may optionally be configured to determine trends of the determined composite respiratory vibrations for each of the two or more segments and determine a change in patient condition using the determined trends, wherein the change in patient condition includes an indication of at least one of: chronic obstructive pulmonary disorder (COPD); asthma; heart failure (HF); pneumonia; bronchitis; or sleep apnea of the patient.

An example (e.g., “Example 12”) of subject matter (e.g., a method) may comprise: receiving, using a signal receiver circuit, physiologic information cyclic with patient respiration for a plurality of respiratory cycles of a patient; receiving, using the signal receiver circuit, vibration information indicative of patient respiratory vibrations for the plurality of respiratory cycles of the patient; identifying, using an assessment circuit, a first set of respiratory cycles of the plurality of respiratory cycles having a duration within a threshold; aligning, using the assessment circuit, segments of the vibration information corresponding to the first set of respiratory cycles, the segments associated with a desired portion of the respiratory cycle using a feature of the respiratory cycle; and determining, using the assessment circuit, a composite respiratory vibration using the aligned segments.

In Example 13, the subject matter of Example 12 may optionally be configured to comprise sensing the vibration information indicative of patient respiratory vibrations using a vibration sensor contained in an implantable housing and storing the determined composite respiratory vibration in a memory in the implantable housing, wherein determining the composite respiratory vibration comprises to improve a signal-to-noise ratio (SNR) of the vibration information.

In Example 14, the subject matter of any one or more of Examples 1-13 may optionally be configured such that the physiologic information and the vibration information comprise different types of information and the physiologic information comprises at least of electrocardiogram information of the patient, accelerometer information of the patient, impedance information of the patient, acoustic information of the patient, or blood flow information of the patient.

In Example 15, the subject matter of any one or more of Examples 1-14 may optionally comprise determining a change in patient status, detecting a physiological condition of the patient, or determining a patient therapy parameter using the determined composite respiratory vibration.

In Example 16, the subject matter of any one or more of Examples 1-15 may optionally be configured such that the first set of respiratory cycles of the patient comprises at least 3 respiratory cycles and the threshold comprises a range within N % of the duration a first respiratory cycle or of the inspiration/expiration (I/E) ratio of the first respiratory cycle.

In Example 17, the subject matter of any one or more of Examples 1-16 may optionally be configured such that the feature of the respiratory cycle comprises a transition between inspiration and expiration of the patient.

In Example 18, the subject matter of any one or more of Examples 1-17 may optionally be configured such that the composite respiratory vibration comprises an average of the aligned vibration information.

In Example 19, the subject matter of any one or more of Examples 1-18 may optionally be configured such that the segments of the vibration information associated with the desired portion of the respiratory cycle comprise segments associated with two or more of: a wheeze segment of the respiratory cycle; a stridor segment of the respiratory cycle; a squawk segment of the respiratory cycle; a rhonchus segment of the respiratory cycle; a snore segment of the respiratory cycle; a fine crackle segment of the respiratory cycle; a course crackle segment of the respiratory cycle; a crackle segment of the respiratory cycle; and a pleural friction rub segment of the respiratory cycle, and the assessment circuit is configured to determine composite respiratory vibrations for each of the two or more segments.

In Example 20, the subject matter of any one or more of Examples 1-19 may optionally comprise determining, using the assessment circuit, trends of the determined composite respiratory vibrations for each of the two or more segments and determining, using the assessment circuit, a change in patient condition using the determined trends, wherein the change in patient condition includes a change in an indication of at least one of: chronic obstructive pulmonary disorder (COPD); asthma; heart failure (HF); pneumonia; bronchitis; or sleep apnea of the patient.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-4 illustrate example respiratory vibrations and respiration signals.

FIG. 5 illustrates an example phase correction circuit.

FIG. 6 illustrates an example phase output of multiple respiration signals.

FIGS. 7-8 illustrate example systems.

FIG. 9 illustrates an example method to determine a composite respiratory vibration.

FIG. 10 illustrates a block diagram of an example machine upon which any one or more of the techniques (e.g., methodologies) discussed herein may perform.

### DETAILED DESCRIPTION

Direct airflow measurements (e.g., direct oronasal airflow measurements) can be obtained using a variety of external sensors or transducers (e.g., external pressure sensors, thermistors, piezoelectric sensors, airflow sensors, etc.) placed on or about the airway of a patient, including in, on, or near a nose or a mouth of the patient, and can include a spirometer, a pressure sensor, a thermistor, a piezoelectric sensor, etc., each in fluid communication (direct contact) with patient airflow into or out of the body. Other traditional external respiration detection can include a belt to detect expansion or contraction of the chest or abdomen associated with respiration. However, expansion and contraction of the chest or abdomen of a patient generally leads direct airflow measurement.

Conceptually, the respiration phase begins with airway muscle movement (e.g., movement of the diaphragm, etc.) and movement of the chest and abdomen, leading to thoracic pressure changes, patient airflow, and respiration sounds. Thoracic pressure changes impact other physiologic information, such as heart rate, arterial pressure, and tissue perfusion.

Respiration phase measurement of a patient can be determined using one or more physiologic signals having respiratory information (e.g., indirect respiration measurements) separate from traditional direct oronasal airflow measurement, such as one or more of an electrocardiogram (ECG) signal, an accelerometer signal, a photoplethysmography (PPG) signal, a transthoracic impedance signal, or one or more other physiologic signals having respiration information for indirect respiration measurement, different than direct airflow measurement. The peaks and valleys of such indirect respiration measurements may lead or lag the peaks and valleys of traditional direct oronasal airflow measurement. Determination of patient respiratory phase information, including inspiration, expiration, or transitions therebetween using such indirect respiration measurements may differ from patient inspiration or expiration determined using direct oronasal airflow. However, different indirect respiration measurements can be aligned using one or more phase correction factors to improve patient respiration phase determination.

An improvement in the accuracy of respiratory phase information can improve the sensitivity of one or more other respiratory parameters, such as inspiration/expiration (I/E) ratio, a forced expiratory volume (FEV) over time (e.g., FEV over 1 second (FEV1)), a forced vital capacity (FVC), respiratory rate (RR), tidal volume (TV), the identification or classification of one or more respiratory vibrations, etc.

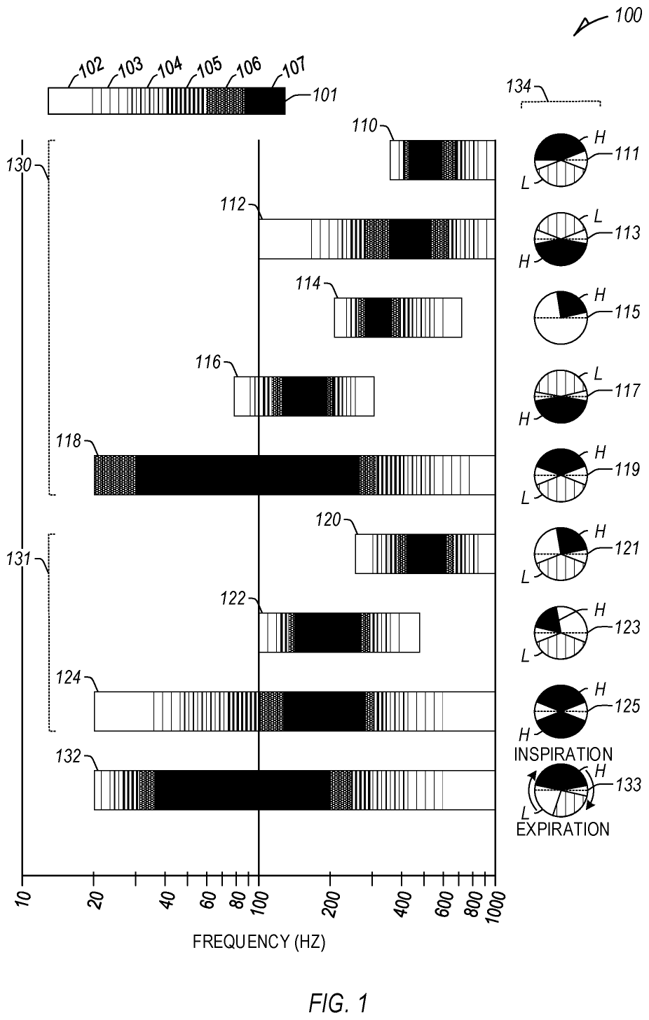

FIG. 1 illustrates example intensities of respiratory vibrations 100, such as respiratory sounds, pressure indicative of vibration, etc., in frequency (Hz) and phase according to a scale 101 varying in intensity level from a first intensity level 102 (less intensity) to a sixth intensity level 107 (more intensity). The respiratory vibrations 100 include continuous sounds 130, discontinuous sounds 131, and normal vesicular breathing 132 ranging in frequency between 20 and 1000 Hz and varying in intensity across different frequencies. In an example, a determined composite respiration phase of the patient can be used to discriminate different respiratory vibrations, such as those illustrated herein, etc. In certain examples, respiratory vibrations can include respiratory sounds, including acoustic or pressure changes indicative of respiratory vibrations.

For example, the continuous sounds 130 include stridor 110 centered about 500 Hz, wheeze 112 centered about 400 Hz, squawk 114 centered about 300 Hz, rhonchus 116 centered about 150 Hz, and snoring 118 centered about 100 Hz. The discontinuous sounds 131 include fine crackles 120 centered about 500 Hz, coarse crackles 122 centered about 200 Hz, and pleural friction rub 124 centered about 200 Hz. Normal vesicular breathing 132 is centered about 100 Hz.

The frequency of a specific respiratory vibration aids identification of the specific sound. However, as nearly all of these sounds have at least some overlap, and in many cases, substantial overlap in frequency range and response, other information is used to aid identification, including, for example, the portion of the respiratory phase in which such specific sound occurs.

Respiratory phase information 134 for each of the respiratory vibrations 100 are illustrated using a circle, the top half (identified using a dashed line) indicating inspiration and the bottom half indicating expiration with transitions between inspiration and expiration at the dashed lines. For example, normal vesicular breathing respiratory phase information 133 indicates more energy during inspiration, for a substantial portion of the inspiration phase, and less energy during the early portion of the expiration phase. In contrast, snoring respiratory phase information 119 indicates more energy during inspiration (similar to normal vesicular breathing 132), and less energy during expiration, but over a larger portion of the expiration than normal vesicular breathing respiratory phase information 133. Thus, snoring 118 and normal vesicular breathing 132, both centered about 100 Hz, can be distinguished using detected differences in respiratory phase information 134. However, more accurate detection of respiratory phase, or respiratory phase changes (between inspiration and expiration), provide more accurate determination of respiratory vibrations 100.

Stridor respiratory phase information 111 indicates more energy earlier in inspiration, and less energy through a longer portion of expiration, than normal vesicular breathing 132. Wheeze respiratory phase information 113 indicates more energy through a large portion of expiration and less energy through a large portion of inspiration. Squawk respiratory phase information 115 indicates more energy during the latter portion of inspiration and little to no energy during expiration. Rhonchus respiratory phase information 117 indicates more energy through a large portion of expiration and less energy through a large portion of inspiration. Snoring respiratory phase information 119 indicates more energy during a large portion of inspiration and less energy during a large portion of expiration. Fine crackle respiratory phase information 121 indicates more energy during the latter portion of inspiration and less energy through a large portion of expiration. Coarse crackle respiratory phase information 123 indicates more energy during the earlier portion of inspiration and less energy through a large portion of expiration. Pleural friction rub respiratory phase information 125 indicates more energy during a large portion of inspiration as well through a large portion of expiration.

Although there is overlap, specific respiratory vibrations or combinations thereof are associated with specific diseases or disease states. For example, chronic obstructive pulmonary disease (COPD) is generally associated with rhonchus, wheeze, and crackles. Heart failure (HF) is generally associated with crackles and wheeze, but not rhonchus. Bronchitis is generally associated with rhonchus and crackles, but not wheeze.

There are a number of challenges in determining diseases or disease states using respiratory vibrations. Individual breaths may contain multiple respiratory vibrations and different respiratory vibrations often resemble each other. In addition, all respiratory vibrations of the patient may not be present in every breath and respective respiratory vibrations can vary from breath to breath for a number of reasons, including voluntary or involuntary breathing changes, speaking or eating, posture, activity, etc. Detection or identification of respiratory vibrations of the patient may require respiratory information from a number of breaths or respiratory cycles.

In an example, respiratory information or vibration information indicative of patient breathing can be received, such as using physiological information from one or more sensors. Individual breaths of the patient can be determined using the received respiratory information or vibration information or one or more other physiologic information of the patient, and a probability of a presence of a plurality of respiratory vibrations can be determined within the determined breaths. Individual breath classifications can be grouped to determine the respiratory vibration for the entire set of individual breaths (cluster).

Tables 1 and 2 illustrate example first and second aggregations. Respiratory sound type probabilities are determined for each of five (5) breaths, such as using a best-fit model of the respiratory vibrations 100 illustrated in FIG. 1. In Table 1, probabilities for each breath do not have to add to 100%, as the individual sound types are not mutually exclusive. As such, the percentages illustrate a probability that a respective breath (e.g., a 1^{st} breath, 2^{nd} breath, 3^{rd} breath, 4^{th} breath, or a 5^{th} breath) illustrates each of the listed conditions, here “Normal”, “Wheeze”, “Snoring”, and “Crackles”. The “Cluster” illustrates the most likely of listed conditions for the group of breaths comprising the 1^{st} breath, 2^{nd} breath, 3^{rd} breath, 4^{th} breath, and a 5^{th} breath.

TABLE 1
First Aggregation

| Sound | 1^{st} | 2^{nd} | 3^{rd} | 4^{th} | 5^{th} | |
|---|---|---|---|---|---|---|
| type\N | breath | breath | breath | breath | breath | Cluster |
| Normal | 75% | 50% | 50% | 25% | 40% | |
| Wheeze | 10% | 55% | 10% | 35% | 0% | |
| Snoring | 35% | 0% | 75% | 0% | 0% | |
| Crackles | 0% | 25% | 25% | 0% | 50% | |
| Most likely | Normal | Wheeze | Snoring | Wheeze | Crackles | Wheeze |

In Table 2, respiratory sound type probabilities are determined for each of five (5) breaths with respect to patient confidence intervals and a threshold probability. Each line in Table 2 is totaled at right, and the highest total is the most likely determined respiratory sound prototype. In other examples, one or more other probabilities or respiratory vibration types can be used.

TABLE 2
Second Aggregation

| | 1^{st} | 2^{nd} | 3^{rd} | 4^{th} | 5^{th} | |
|---|---|---|---|---|---|---|
| Sound type\N | breath | breath | breath | breath | breath | Total |
| Normal (1 if | 1 | 0 | 0 | 0 | 0 | 1 |
| P >= 75%) | | | | | | |
| Wheeze (1 if | 0 | 1 | 0 | 1 | 0 | 2 |
| P >= 25%) | | | | | | |
| Snoring (1 if | 0 | 0 | 1 | 0 | 0 | 1 |
| P >= 50%) | | | | | | |
| Crackles (1 if | 0 | 1 | 1 | 0 | 1 | 3 |
| P >= 25%) | | | | | | |
| Most likely | | | | | | Crackles |

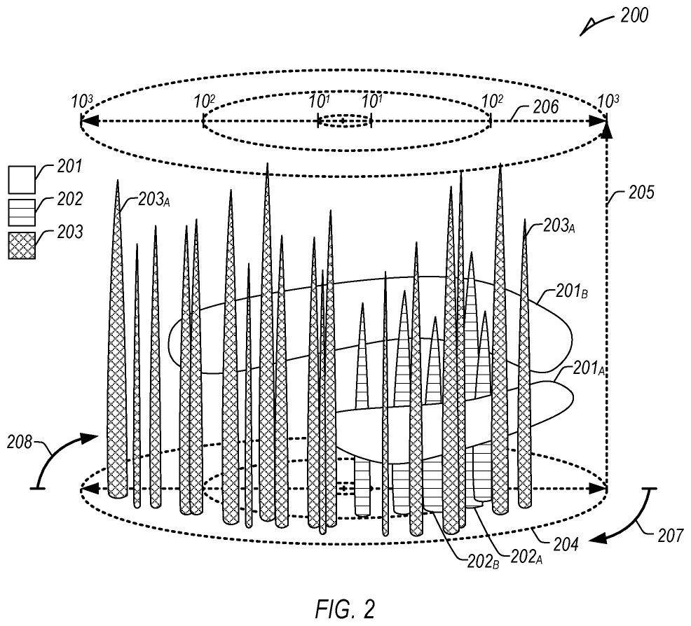

FIG. 2 illustrates generally a third aggregation of multi-dimensional respiratory information 200 over multiple (e.g., 5) respiration cycles, including type 1 information 201, type 2 information 202, and type 3 information 203 with respect to multiple dimensions, including respiration phase 204, respiration intensity 205, and respiration frequency 206. In other examples, one or more other sets of dimensions can be used to visualize the respiratory sounds, such as two or more of duration, intensity, rapidity of onset, rate/consistency of occurrence, etc., separate from or in combination with the dimensions illustrated in FIG. 2.

The respiration phase can 204 be visualized as a 360° circular path around the center/base of the display, including the start of inspiration 207 and the start of expiration 208. The respiration frequency can be visualized as low at the origin/center of the display, increasing towards the outer ring. The respiration intensity 205 can be visualized as the vertical travel or magnitude of the respiratory information 200. Clusters of sounds with similar characteristics can be identified and displayed, and changes in patient sounds over time can be an indicator of patient status or a change in patient status.

The type 1 information 201 can include continuous sounds from 300-500 HZ, similar to a wheeze, favoring early inspiration at 201A and expiration at 201B. The type 2 information 202 can include discontinuous sounds from 100-300 Hz, similar to a coarse crackle, favoring early inspiration at 202_{A}, 202_{B}. The type 3 information 203 can include discontinuous sounds from 400-600 Hz, favoring inspiration 603.

Grouping respiratory vibration information over multiple respiration cycles and sorting by respiratory phase, amplitude, and frequency, can help detect and identify repeating respiratory vibrations with a higher confidence and accuracy than detecting and identifying sounds in single respiration cycles. Further, in certain examples, composite respiratory vibrations can be determined, such as by averaging or otherwise combining similar measurements of respiratory vibrations into a composite measurement. For example, respiratory cycles having a duration (e.g., an inspiration duration, an expiration duration, a respiratory cycle duration, etc.) within a threshold (e.g., substantially similar cycle duration, such as within 5%, 10%, etc.) can be identified. Segments of the identified respiratory vibrations can be selected, such as corresponding to one or more respiratory vibrations identified in FIG. 1, etc. The selected segments can be aligned, such as with respect to a determined respiration phase or one or more other respiratory markers, etc., and the aligned selected segments can be combined (e.g., averaged, etc.) into a composite segment, such as to reduce noise, to improve signal-to-noise ratio (SNR), to capture respiratory vibrations that may not be present in every cycle, to reduce cycle-to-cycle variation, to reduce storage requirements, etc. A single composite segment or measure can contain the information of a number of respiratory measures, and can be more indicative of patient status than the individual measures that go into it. Further, changes in the composite measure or segment over time, such as long or short-term changes, short-term changes with respect to long-term changes, deviation of a daily measure from a baseline, etc., can be indicative of changing patient status, and accordingly used to determine a measure of changing patient status, etc.

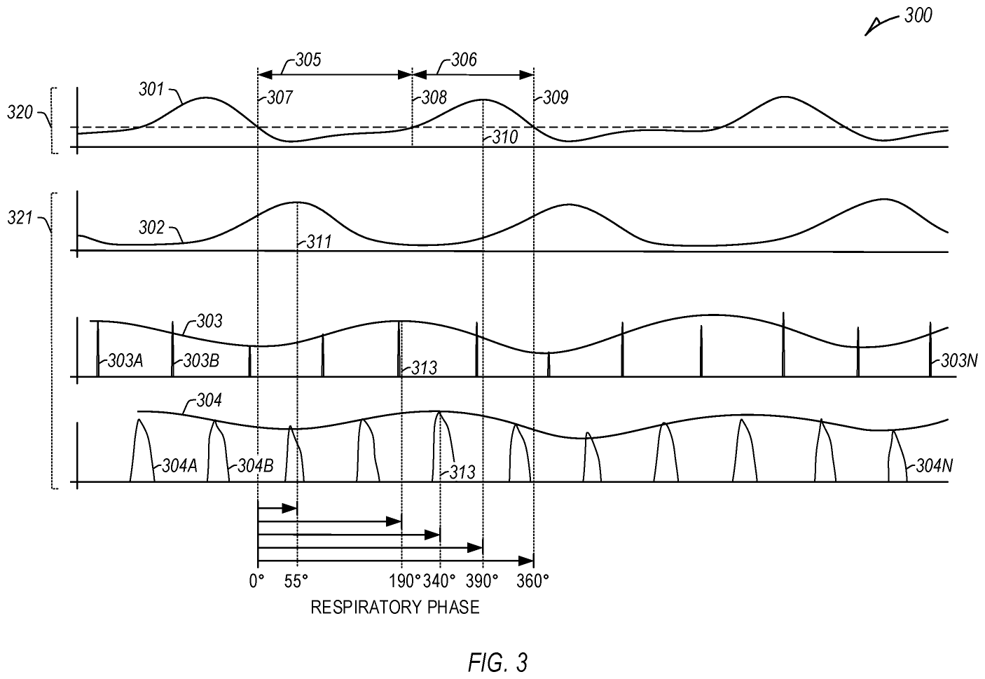

FIG. 3 illustrates example respiratory phase differences 300 between different respiration signals including a direct respiration measurement 320 and several indirect respiration measurements 321. The direct respiration measurement 320 includes an oronasal airflow signal 301 (e.g., an oral-nasal pressure signal). The indirect respiration measurements 321 include an impedance respiration signal 302 (e.g., an impedance pneumography), an ECG respiration signal 303 (determined using R-wave peaks 303A-N of an ECG signal), and a PPG respiration signal 304 (determined using PPG peaks 304A-N of a PPG signal).

Periods of inspiration 305 and expiration 306 are marked at zero-crossings of the oronasal airflow signal 301, including a first zero-crossing 307 marking a transition from positive airflow to negative airflow (the beginning of inspiration 305), a second zero-crossing 308 marking a transition from negative airflow to positive airflow (the end of inspiration 305 and beginning of expiration 306), and a third zero-crossing 309 marking a transition from positive airflow to negative airflow (the end of expiration 306 and beginning of a subsequent respiratory phase).

The challenges in accurately determining respiratory phase are multifaceted. One way to determine respiratory phase information from indirect respiration measurements 321, such as physiologic signals having a respiratory component, is by detecting peaks in components of the physiologic signal having respiratory information. For example, the ECG respiration signal 303 illustrated in FIG. 3 is determined using an average R-wave amplitude of multiple R-wave peaks 303A-N of ECG information of a patient, and the PPG respiration signal 304 is determined using an average PPG signal amplitude of multiple PPG peaks 304A-N of PPG information of the patient.

Certain respiratory parameters, such as respiration frequency, can be determined using the number and location of detected peaks. However, the peaks often do not uniformly correspond to a specific portion of a respiratory phase. For example, with respect to the first zero-crossing 307 corresponding to inspiration 305 being 0° of a respiratory phase (having 360° between successive inspiration periods), an impedance peak 311 is at 55°, an ECG peak 312 is at 190°, a PPG peak 313 is at 340°, an oronasal peak 310 is at 290°. The third zero-crossing 309 closes the respiratory phase at 360°. As there are no indirect respiration measurement peaks about 0°/360°, such transition (the first or third zero-crossing 307, 309) is often estimated following one or more other detected peaks. The second zero-crossing 308 occurs at approximately 200° (different than the illustrated 180° respiratory phase information 134 in FIG. 1). Although the second zero-crossing relatively closely follows the ECG peak 312, estimation is again often required.

The challenge is amplified in that inspiration and expiration can have different period lengths, with inspiration 305 often longer than expiration 306. Further, in certain examples, one or both of peaks and troughs can be difficult to identify. For example, the impedance respiration signal 302 has a defined peak (e.g., at impedance peak 311), but the trough is more difficult to identify. In certain examples, the signal can be inverted, such as depending on sensor (e.g., one or more electrodes) polarity, placement, or one or more other factors. Accurate detection of periods of inspiration 305 and expiration 306 are important, such as to detect the I/E ratio, classification of respiratory sounds, etc. Further, in certain examples, respiratory phase information can be scaled for display to reflect desired information. For example, the period lengths of inspiration 305 and expiration 306 in FIG. 3, approximately 200° and 160°, respectively, can be scaled to reflect equal phase distributions of 180°, such as illustrated in the respiratory phase information 134 of FIG. 1.

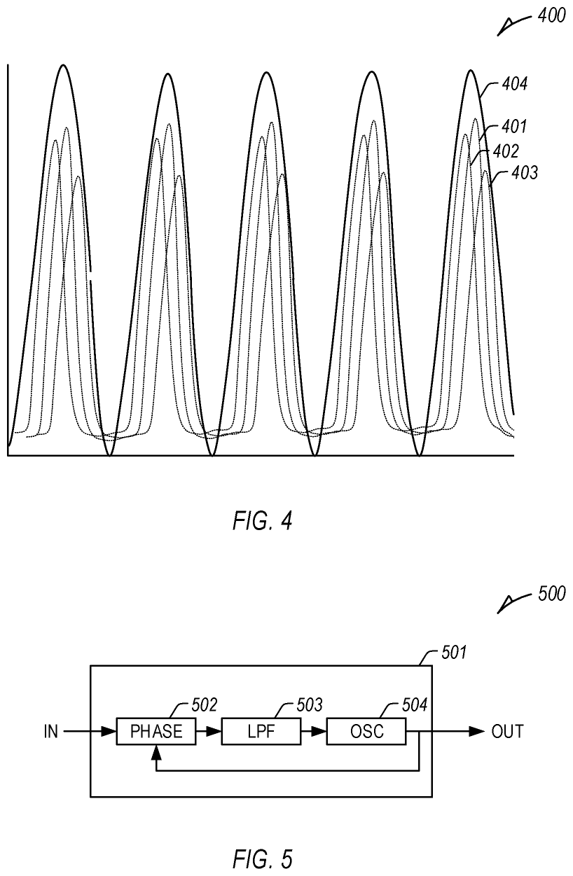

FIG. 4 illustrates example respiration signals 400 having different phase shifts, including first, second, and third respiration signals 401, 402, 403, and a composite, corrected respiration signal 404. In an example, the first respiration signal 401 can include an impedance respiration signal, the second respiration signal 402 can include an ECG respiration signal, and the third respiration signal 403 can include a PPG respiration signal. In other examples, the respiration signals 400 can include one or more other physiologic signals having other physiologic signals having a respiratory component.

Phase correction factors can be used to align respiration measurements to the actual respiration phase of the patient (e.g., the phase of a direct measurement of patient airflow). Such phase correction factors can be population-based, patient-specific, or combinations thereof. To increase signal integrity, such as in the presence of noise, a composite of multiple physiologic signals having respiratory components can be combined, for example, after alignment using one or more correction factors. In an example, one or more phase lock loop (PLL) circuits, such as of a medical device or one or more other components associated with a medical-device system, can be used to align the multiple physiologic signals.

In a similar manner, a composite respiratory vibration (e.g., 5 respiratory cycles or more, etc.) can be obtained by first grouping and aligning breaths, such as based on a respiration phase timing, and then aggregating vibration information and classifying its type. Such a composite approach can improve the accuracy and robustness of breathing sound classification.

FIG. 5 illustrates an example phase correction circuit 500 including a phase lock loop (PLL) circuit 501 configured to receive an input signal (IN) (e.g., physiologic information, such as from a physiologic signal having imperfect respiratory information, etc.) and provide an output signal (OUT). In an example, the input signal can often be noisy, unstable, or non-sinusoidal. The PLL circuit 501 can be configured to provide an output signal, based on the input signal, having a clear, stable frequency and sinusoidal phase.

The PLL circuit 501 include a phase comparator (PHASE) circuit 502, a low-pass filter (LPF) circuit 503, and an oscillator (OSC) circuit 504. The phase comparator circuit 502 can ensure that the output signal maintains a relatively consistent phase angle in relation to the input signal, such as by determining a phase difference between the input signal and the output signal and providing an output signal representative of the difference. The low-pass filter 503 can filter high frequency noise from the output of the phase comparator circuit 502. The oscillator circuit 504 (e.g., an amplitude-controlled oscillator, etc.) can receive the filtered output of the low-pass filter 503 and provide an output signal (e.g., a sinusoidal output signal) having a frequency controlled by the output of the low-pass filter 503.

In certain examples, the phase correction circuit 500 or the PLL circuit 501 can include one or more other components or circuits. In an example, the PLL circuit 501 can include a loop filter circuit or one or more other circuits or components configured to control the feedback from the output of the oscillator circuit 504 to the phase comparator circuit 502, such as to control the stability of the loop, the speed or responsiveness of the loop, etc. Although described and illustrated herein as a sinusoidal output signal, in other examples, the output signal (OUT) can take one or more other shapes or forms, such as a square-wave output, a sawtooth-wave output, etc.

In certain examples, the PLL circuit 501 can receive a respiration signal (e.g., a non-sinusoidal respiration signal), such as one or more of the respiration signals 400 of FIG. 4, and provide a sinusoidal output, such as an output signal 404 illustrated in FIG. 4, for each received respiration signal, or for a composite of multiple respiration signals or received patient respiration information.

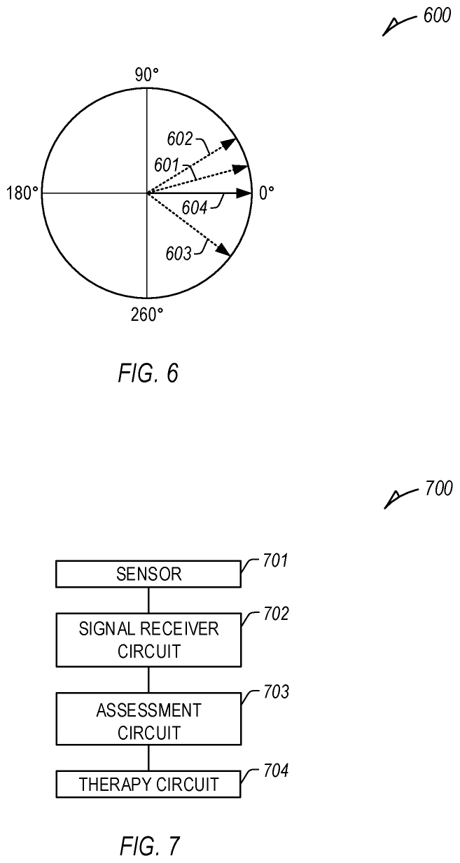

FIG. 6 illustrates an example phase output 600 of multiple respiration signals, such as the respiration signals 400 illustrated in FIG. 4. The phase output 600 can include first, second, third, and fourth phase outputs 601-604. In an example, the fourth phase output 604 can be indicative of a phase of an output signal, or a combination or composite of multiple respiration signals, such as the first, second, and third respiration signals 401, 402, 403 of FIG. 4.

The first phase output 601 can be indicative of a phase of a first respiration signal, such as of the first respiration signal 401 of FIG. 4, with respect to the fourth phase output 604 (0°). The second phase output 602 can be indicative of a phase of a second respiration signal, such as the second respiration signal 402 of FIG. 4, with respect to the fourth phase output 604. The third phase output 603 can be indicative of a phase of a third respiration signal, such as the third respiration signal 403 of FIG. 4, with respect to the fourth phase output 604.

In certain examples, the direction and delay of each of the respiration signals can be patient specific. In one example, although variable, phase shifts for certain parameters, with respect to direct measurement of patient airflow (0°), can be expected as illustrated in Table 1 (with positive numbers indicating phase lag).

TABLE 3
Example Respiration Phase Shift

| Physiologic | Chest | ECG R Wave | ECG Heart | | Arterial |
|---|---|---|---|---|---|
| Signal | Movement | Amplitude | Rate | PPG | Pressure |
| Phase Shift | −70° | 0° | +55° | +85° | +50° |
| Relative to | | | | | |
| Airflow | | | | | |

In an example, for implantable medical devices, a common signal fiducial (e.g., a peak value, a zero-cross value, etc.) can be identified or measured at implant or programming to determine phase shift among different signals. In certain examples, signal fiducials can be identified or measured at implant or programming at different controlled configurations or situations (e.g., different postures, different activity levels, etc.) or during different doses of a therapy (e.g. medication, continuous positive airway pressure (CPAP) or other respiratory therapy, cardiac pacing, neuro-modulation) wherein the different dose might include no dose of a therapy. Initial values or measurements can be used to control combination of different physiologic signals, or as an initial data point for later combination or adjustment.

In certain examples, the direction and delay of respiration phase shifts can be used to track or determine patient health status or changes in patient health, such as disease state changes, etc. For example, modulation of physiologic information due to respiration can decrease due to fluid overflow or shallow breathing. Accordingly, decreased phase shifts of patient physiologic information relative to patient airflow can be indicative of heart failure, or a worsening or change in heart failure status. Modulation of physiologic information due to respiration can increase due to airway obstruction or increased thoracic pressure. Accordingly, increased phase shifts of patient physiologic information relative to patient airflow can be indicative of chronic obstructive pulmonary disease (COPD) or asthma, or a worsening or change in COPD or asthma status.

In an example, the direction and delay of respiration phase shifts can be used to track or determine therapy effectiveness, such as a medication therapy, continuous positive airway pressure or other respiratory therapy, cardiac pacing therapy, or neuro-modulation therapy. For example, nebulizer therapy can be used to reduce airway congestion associated with COPD or asthma. Reduced phase shifts of patient physiologic information relative to patient airflow can be indicative of improvement in airway congestion due to effective nebulizer therapy.

Modulation of physiologic information due to respiration can cease during an apnea event. Accordingly, severe and unstable phase shifts for several (e.g., 5-10) respiration cycles after resolution of the apnea can be indicative of an apnea event. Modulation of physiologic information due to respiration can decrease during a hypopnea event. Accordingly, moderate but unstable phase shifts for several (e.g., 5-10) respiration cycles can be indicative of a hypopnea event, or resolution of a hypopnea event.

Modulation of physiologic information due to respiration can decrease due to shallow breathing, such as indicative of patient pneumonia. Accordingly, decreased phase shifts of patient physiologic information relative to patient airflow can be indicative of shallow breathing, patient pneumonia, or a worsening or change in shallow breathing or patient pneumonia.

FIG. 7 illustrates an example system 700, such as a medical-device system, etc. In an example, one or more aspects of the example system 700 can be a component of, or communicatively coupled to, an ambulatory medical device (AMD). AMDs can be configured to monitor, detect, or treat various physiologic conditions of the body, such as cardiac conditions associated with a reduced ability of a heart to sufficiently deliver blood to a body, including HF, arrhythmias, hypertension, dyssynchrony, etc. AMDs can include a single device or a plurality of medical devices or monitors implanted in a patient's body or otherwise positioned on or about the patient to monitor patient physiologic information of the patient, such as using one or more sensors, the physiologic information including one or more of heart sounds, respiration (e.g., respiration rate, tidal volume (TV), etc.), respiration sounds, impedance (e.g., thoracic impedance, cardiac impedance, cutaneous impedance, etc.), pressure (e.g., blood pressure), cardiac activity (e.g., heart rate, cardiac electrical information, etc.), chemical (e.g., electrolyte), physical activity, posture, plethysmography, or one or more other physiologic parameters of a patient, or to provide electrical stimulation or one or more other therapies or treatments to the patient.

The example system 700 can include a signal receiver circuit 702 and an assessment circuit 703. The signal receiver circuit 702 can be configured to receive physiologic information of a patient (or group of patients) from one or more sensors 701. The assessment circuit 703 can be configured to receive information from the signal receiver circuit 702, and to determine one or more parameters (e.g., physiologic parameters, stratifiers, etc.) or existing or changed patient conditions (e.g., indications of patient dehydration, a respiratory condition (e.g. chronic obstructive pulmonary disease (COPD), asthma), a cardiac condition (e.g. heart failure, arrhythmia), etc.) using the received physiologic information, such as described herein. The physiologic information can include, among other things, cardiac electrical information, impedance information, respiration information, heart sound information, activity information, posture information, temperature information, chemical information, etc.

In an example, the sensor 701 can include one or more of: a respiration sensor configured to receive respiration information (e.g., a respiration rate, a respiration volume (tidal volume), respiratory vibrations, vibration sounds indicative of respiratory sounds, etc.); an acceleration sensor (e.g., an accelerometer, a microphone, a hydrophone, a vibration sensor, etc.) configured to receive cardiac or other acceleration information (e.g., cardiac vibration information, pressure waveform information, heart sound information, respiration information, endocardial acceleration information, acceleration information, activity information, posture information, etc.); an acoustic sensor (e.g. a microphone, a hydrophone) configured to receive cardiac, respiratory or other physiological sounds, an impedance sensor (e.g., intrathoracic impedance sensor, transthoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a blood gas, etc.); a skin temperature sensor; a skin elasticity sensor, or one or more other sensors configured to receive physiologic information of the patient.

The assessment circuit 703 can be configured to provide an output to a user, such as to a display or one or more other user interface, the output including a score, a trend, an alert, or other indication. In other examples, the assessment circuit 703 can be configured to provide an output to another circuit, machine, or process, such as a therapy circuit 704 (e.g., a cardiac resynchronization therapy (CRT) circuit, a chemical therapy circuit, etc.), etc., to control, adjust, or cease a therapy of a medical device, a drug delivery system, etc., or otherwise alter one or more processes or functions of one or more other aspects of a medical-device system, such as one or more CRT parameters, drug delivery, dosage determinations or recommendations, etc. In an example, the therapy circuit 704 can include one or more of a stimulation control circuit, a cardiac stimulation circuit, a neural stimulation circuit, a dosage determination or control circuit, etc. In other examples, the therapy circuit 704 can be controlled by the assessment circuit 703, or one or more other circuits, etc.

AMDs can include a range of medical devices, including, for example, traditional cardiac rhythm management (CRM) devices, such as pacemakers, defibrillators, or cardiac resynchronizers, include implantable or subcutaneous devices configured to be implanted in a chest of a patient. The CRM device can include one or more leads to position one or more electrodes or other sensors at various locations in or near the heart, such as in one or more of the atria or ventricles. Separate from, or in addition to, the one or more electrodes or other sensors of the leads, the CRM device can include one or more electrodes or other sensors (e.g., a pressure sensor, an accelerometer, a gyroscope, a microphone, etc.) powered by a power source in the CRM device. The one or more electrodes or other sensors of the leads, the CRM device, or a combination thereof, can be configured detect physiologic information from the patient, or provide one or more therapies or stimulation to the patient.

Implantable devices can additionally or separately include leadless cardiac pacemakers (LCP), small (e.g., smaller than traditional implantable CRM devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable CRM device complications (e.g., required incision and pocket, complications associated with lead placement, breakage, or migration, etc.). In certain examples, an LCP can have more limited power and processing capabilities than a traditional CRM device; however, multiple LCP devices can be implanted in or about the heart to detect physiologic information from, or provide one or more therapies or stimulation to, one or more chambers of the heart. The multiple LCP devices can communicate between themselves, or one or more other implanted or external devices.

Each additional sensor within or associated with an AMD or medical device system can increase system cost and complexity, reduce system reliability, or increase the power consumption and reduce the usable life of the AMD.

Accordingly, it can be beneficial to use a single sensor to determine multiple types of physiologic information, or a smaller number of sensors to measure a larger number of different types of physiologic information. For example, it can be beneficial to detect atrial cardiac electrical information without a lead or an electrode in, or in contact with, the atria. Similarly, it can be beneficial to detect accurate respiration phase information without a direct measurement of patient airflow.

FIG. 8 illustrates an example patient management system 800 and portions of an environment in which the system 800 may operate. The patient management system 800 can perform a range of activities, including remote patient monitoring and diagnosis of a disease condition. Such activities can be performed proximal to a patient 801, such as in a patient home or office, through a centralized server, such as in a hospital, clinic, or physician office, or through a remote workstation, such as a secure wireless mobile computing device.

The patient management system 800 can include one or more AMDs, an external system 805, and a communication link 811 providing for communication between the one or more AMDs and the external system 805. The one or more AMDs can include an implantable medical device (IMD) 802, a wearable medical device 803, or one or more other implantable, leadless, subcutaneous, external, wearable, or AMDs configured to monitor, sense, or detect information from, determine physiologic information about, or provide one or more therapies to treat various conditions of the patient 801, such as one or more cardiac or non-cardiac conditions (e.g., dehydration, etc.).

In an example, the IMD 802 can include one or more traditional cardiac rhythm management (CRM) devices, such as a pacemaker or defibrillator, implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 801. In another example, the IMD 802 can include a monitor implanted, for example, subcutaneously in the chest of patient 801.

The IMD 802 can include an assessment circuit configured to detect or determine specific physiologic information of the patient 801, or to determine one or more conditions or provide information or an alert to a user, such as the patient 801 (e.g., a patient), a clinician, or one or more other caregivers or processes. The IMD 802 can alternatively or additionally be configured as a therapeutic device configured to treat one or more medical conditions of the patient 801. The therapy can be delivered to the patient 801 via the lead system and associated electrodes or using one or more other delivery mechanisms. The therapy can include delivery of one or more drugs to the patient 801 using the IMD 802 or one or more of the other AMDs. In some examples, therapy can include CRT for rectifying dyssynchrony and improving cardiac function in CHF patients. In other examples, the IMD 802 can include a drug delivery system, such as a drug infusion pump to deliver drugs to the patient for managing arrhythmias or complications from arrhythmias, hypertension, or one or more other physiologic conditions.

The wearable medical device 803 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.). The wearable medical device 803 can include an optical sensor configured to detect a PPG signal on a wrist, finger, or other location on the patient 801. In other examples, the wearable medical device 803 can include an acoustic sensor or accelerometer to detect acoustic information (e.g., heart sounds) or the sound or vibration of blood flow, an impedance sensor to detect impedance variations associated with changes in blood flow or volume, a temperature sensor to detect temperature variation associated with blood flow, a laser Doppler vibrometer or other pressure, strain, or physical sensor to detect physical variations associated with blood flow, etc.

The patient management system 800 can include, among other things, a respiration sensor configured to receive respiration information (e.g., a respiration rate, a respiration volume (a minute volume (MV), a tidal volume (TV), etc.), etc.), a heart sound sensor configured to receive heart sound information, a thoracic impedance sensor configured to receive impedance information, a cardiac sensor configured to receive cardiac electrical information, an activity sensor configured to receive information about a physical motion (e.g., activity, posture, etc.), a plethysmography sensor, or one or more other sensors configured to receive physiologic information of the patient 801.

The external system 805 can include a dedicated hardware/software system, such as a programmer, a remote server-based patient management system, or alternatively a system defined predominantly by software running on a standard personal computer. The external system 805 can manage the patient 801 through the IMD 802 or one or more other AMDs connected to the external system 805 via a communication link 811. In other examples, the IMD 802 can be connected to the wearable device 803, or the wearable device 803 can be connected to the external system 805, via the communication link 811. This can include, for example, programming the IMD 802 to perform one or more of acquiring physiologic data, performing at least one self-diagnostic test (such as for a device operational status), analyzing the physiologic data to detect a cardiac arrhythmia, or optionally delivering or adjusting a therapy to the patient 801. Additionally, the external system 805 can send information to, or receive information from, the IMD 802 or the wearable device 803 via the communication link 811. Examples of the information can include real-time or stored physiologic data from the patient 801, diagnostic data, such as detection of patient hydration status, hospitalizations, responses to therapies delivered to the patient 801, or device operational status of the IMD 802 or the wearable device 803 (e.g., battery status, lead impedance, etc.). The communication link 811 can be an inductive telemetry link, a capacitive telemetry link, or a radio-frequency (RF) telemetry link, or wireless telemetry based on, for example. “strong” Bluetooth or IEEE 802.11 wireless fidelity “Wi-Fi” interfacing standards. Other configurations and combinations of patient data source interfacing are possible.

By way of example and not limitation, the external system 805 can include an external device 806 in proximity of the one or more AMDs, and a remote device 808 in a location relatively distant from the one or more AMDs, in communication with the external device 806 via a communication network 807. Examples of the external device 806 can include a medical device programmer.

The remote device 808 can be configured to evaluate collected patient or patient information and provide alert notifications, among other possible functions. In an example, the remote device 808 can include a centralized server acting as a central hub for collected data storage and analysis. The server can be configured as a uni-, multi-, or distributed computing and processing system. The remote device 808 can receive data from multiple patients. The data can be collected by the one or more AMDs, among other data acquisition sensors or devices associated with the patient 801. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more AMDs, such as the IMD. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 808 may additionally include one or more locally configured clients or remote clients securely connected over the communication network 807 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 808, including the server and the interconnected clients, may also execute a follow-up scheme by sending follow-up requests to the one or more AMDs, or by sending a message or other communication to the patient 801 (e.g., the patient), clinician or authorized third party as a compliance notification.

The communication network 807 can provide wired or wireless interconnectivity. In an example, the communication network 807 can be based on the Transmission Control Protocol/Internet Protocol (TCP/IP) network communication specification, although other types or combinations of networking implementations are possible. Similarly, other network topologies and arrangements are possible.

One or more of the external device 806 or the remote device 808 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 806 or the remote device 808 can include a respective display unit for displaying the physiologic or functional signals, or alerts, alarms, emergency calls, or other forms of warnings to signal the detection of arrhythmias. In some examples, the external system 805 can include an external data processor configured to analyze the physiologic or functional signals received by the one or more AMDs, and to confirm or reject the detection of arrhythmias. Computationally intensive algorithms, such as machine-learning algorithms, can be implemented in the external data processor to process the data retrospectively to detect cardia arrhythmias.

Portions of the one or more AMDs or the external system 805 can be implemented using hardware, software, firmware, or combinations thereof. Portions of the one or more AMDs or the external system 805 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The patient management system 800 can include a therapy device 810, such as a respiratory therapy device (e.g. continuous positive airway pressure device or nebulizer device, etc.) or a drug delivery device configured to provide therapy or therapy information (e.g., dosage information, etc.) to the patient 801, such as using information from one or more of the AMDs. In other examples, one or more of the AMDs can be configured to provide therapy or therapy information to the patient 801. The therapy device 810 can be configured to send information to or receive information from one or more of the AMDs or the external system 805 using the communication link 811. In an example, the one or more AMDs, the external device 806, or the remote device 808 can be configured to control one or more parameters of the therapy device 810.

The external system 805 can allow for programming the one or more AMDs and can receives information about one or more signals acquired by the one or more AMDs, such as can be received via a communication link 811. The external system 805 can include a local external IMD programmer. The external system 805 can include a remote patient management system that can monitor patient status or adjust one or more therapies such as from a remote location.

The assessment circuit may be implemented at the external system 805, which can be configured to perform HF risk stratification such as using data extracted from the one or more AMDs or data stored in a memory within the external system 805. Portions of patient chronic condition-based HF or other assessment circuit may be distributed between the one or more AMDs and the external system 805.

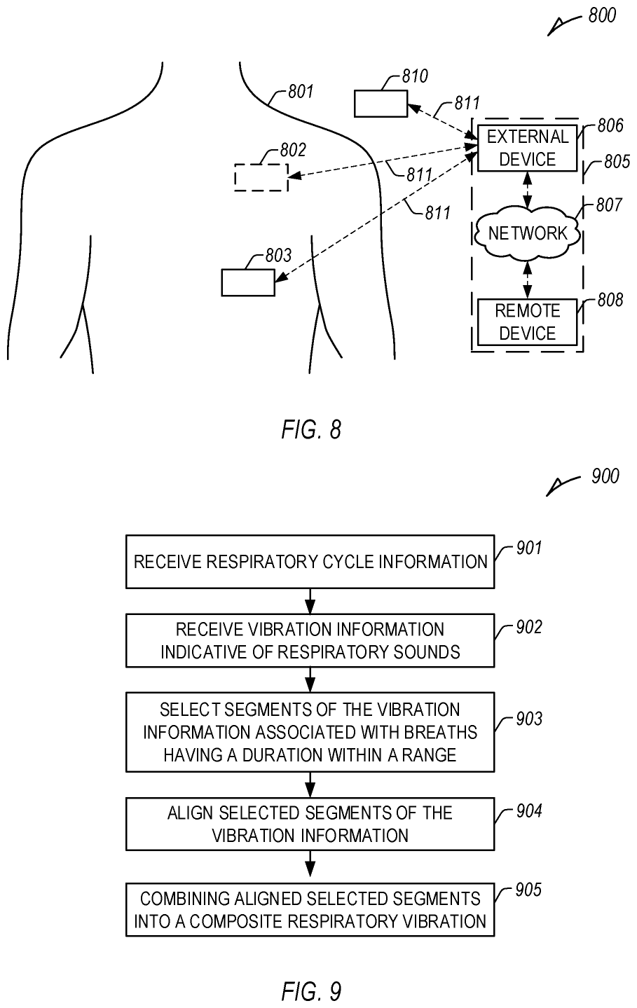

FIG. 9 illustrates an example method 900 to determine a composite respiratory vibration (e.g., a composite respiratory sound) of a patient using received first and second physiologic information of the patient. At 901, respiratory cycle information of the patient can be received, such as physiologic information cyclic with patient respiration for a plurality of respiratory cycles of a patient, such as at a signal receiver circuit of a system, using as a medical device system. The physiologic information can include indirect respiration measurements, or include physiologic information having a respiration component. In certain examples, one or both of the first and second physiologic information can include physiologic signals having a respiration component (e.g., having a component cyclic with patient respiration, etc.), such as one or more of an electrocardiogram (ECG) signal, an accelerometer signal, a vibration signal, an acoustic signal, or a photoplethysmography (PPG) signal, etc. In other examples, additional physiologic information can be received.

The first physiologic information can be different than the second physiologic information. The first physiologic information can include information from a first physiologic signal over a first period, and the second physiologic information can include information from a second physiologic signal over a second period. In certain examples, each of the first and second periods can include at least a portion of a respiratory cycle of the patient. In other examples, each of the first and second periods can include at least one full respiratory cycle of the patient. In an example, the second period can at least partially overlap the first period. In other examples, the first period can be the same as the second period, or can correspond to the same or overlapping respiration cycles of the patient.

At 902, vibration information indicative of respiratory vibrations, including, for example, respiratory sounds, can be received, such as using the signal receiver circuit. The vibration information can include acoustic information, vibration information, or acceleration information sensed, in certain examples, using a sensor contained in a housing of an implantable medical device (IMD). In certain examples, the received vibration information can include vibration information for the plurality of respiratory cycles corresponding to the plurality of respiratory cycles of the patient in step 901. A respiration phase can include information indicative of inspiration and expiration of the patient, including, for example, transitions between inspiration and expiration of multiple respiration phases (e.g., sequential respiration phases).

At 903, segments of the vibration information associated with breaths or respiratory cycles having a duration within a range are selected, such as using an assessment circuit. In an example, a first set of respiratory cycles (e.g., 3, 5, 7, 9, etc.) of the plurality of respiratory cycles having a similar duration can be identified, such as ensuring that the set of respiratory cycles all have durations within a threshold amount or range, or within a threshold amount of each other. In an example, the threshold can include a range within a portion or percentage (e.g., N %) of the duration of the respiratory cycle (e.g., 5%, 10%, etc.) or of the inspiration/expiration (I/E) ratio of the first respiratory cycle, etc. In other examples, the first set can include a number of the plurality of respiratory cycles (e.g., 3, 5, etc.) closest in duration.

In an example, segments of the vibration information associated with the desired portion of the respiratory cycle can include segments associated with two or more of: a wheeze segment of the respiratory cycle; a stridor segment of the respiratory cycle; a squawk segment of the respiratory cycle; a rhonchus segment of the respiratory cycle; a snore segment of the respiratory cycle; a fine crackle segment of the respiratory cycle; a course crackle segment of the respiratory cycle; a crackle segment of the respiratory cycle; and a pleural friction rub segment of the respiratory cycle. In certain examples, composite respiratory vibrations can be determined for each of the two or more segments.

Trends of the determined composite respiratory vibrations can be determined. Changes in patient condition can be detected using the determined trends. In certain examples, changes in patient condition can include a change in an indication of at least one of: chronic obstructive pulmonary disorder (COPD); asthma; heart failure (HF); pneumonia; bronchitis; or sleep apnea of the patient.

At 904, selected segments of the vibration information can be aligned, such as using the assessment circuit. The selected segments can be associated with a desired portion of the respiratory cycle, such as one or more portions associated with one or more respiratory vibrations. The selected segments can be aligned using a feature of the respiratory cycle, such as a beginning of inspiration, a beginning of expiration, a transition between inspiration and expiration, or one or more other markers of the respiratory cycle, for example, determined using the physiologic information cyclic with patient respiration.

At 905, the aligned selected segments can be combined into a composite respiratory vibration, such as using the assessment circuit. In certain examples, the assessment circuit is contained in an IMD, and the determined composite respiratory vibration can be stored in a memory in the IMD. The composite respiratory vibration can have an improved signal-to-noise ratio (SNR), such as compared to the received vibration information alone.

In certain examples, one or more of a change in patient status, a physiological condition of the patient, or a patient therapy parameter can be determined or detected using the determined composite respiratory vibration.

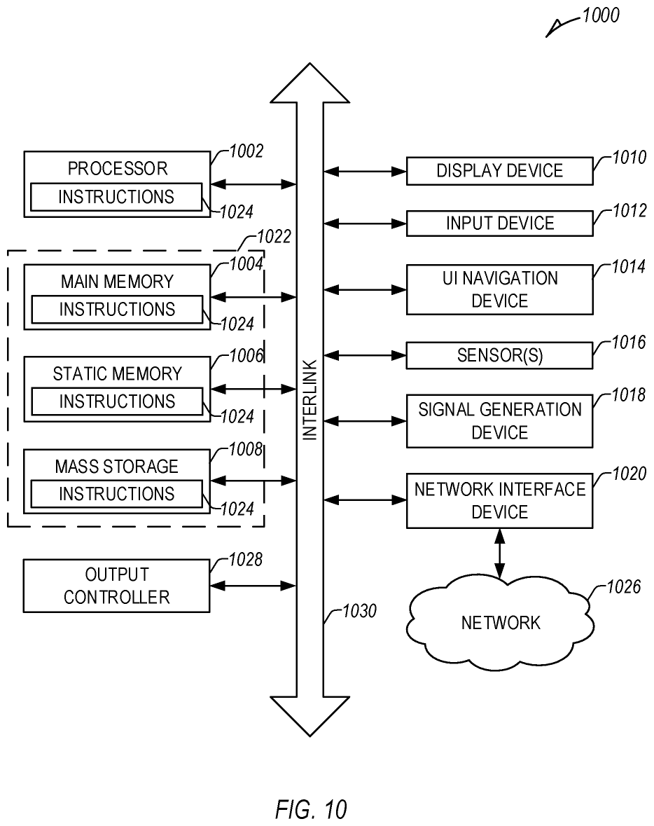

FIG. 10 illustrates a block diagram of an example machine 1000 upon which any one or more of the techniques (e.g., methodologies) discussed herein may perform. Portions of this description may apply to the computing framework of one or more of the medical devices described herein, such as the IMD, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1000. Circuitry (e.g., processing circuitry, an assessment circuit, etc.) is a collection of circuits implemented in tangible entities of the machine 1000 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1000 follow.

In alternative embodiments, the machine 1000 may operate as a standalone device or may be connected (e.g., networked) to other machines. In a networked deployment, the machine 1000 may operate in the capacity of a server machine, a client machine, or both in server-client network environments. In an example, the machine 1000 may act as a peer machine in peer-to-peer (P2P) (or other distributed) network environment. The machine 1000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1000 may include a hardware processor 1002 (e.g., a central processing unit (CPU), a graphics processing unit (GPU), a hardware processor core, or any combination thereof), a main memory 1004, a static memory (e.g., memory or storage for firmware, microcode, a basic-input-output (BIOS), unified extensible firmware interface (UEFI), etc.) 1006, and mass storage 1008 (e.g., hard drive, tape drive, flash storage, or other block devices) some or all of which may communicate with each other via an interlink (e.g., bus) 1030. The machine 1000 may further include a display unit 1010, an alphanumeric input device 1012 (e.g., a keyboard), and a user interface (UI) navigation device 1014 (e.g., a mouse). In an example, the display unit 1010, input device 1012, and UI navigation device 1014 may be a touch screen display. The machine 1000 may additionally include a signal generation device 1018 (e.g., a speaker), a network interface device 1020, and one or more sensors 1016, such as a global positioning system (GPS) sensor, compass, accelerometer, or one or more other sensors. The machine 1000 may include an output controller 1028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1002, the main memory 1004, the static memory 1006, or the mass storage 1008 may be, or include, a machine-readable medium 1022 on which is stored one or more sets of data structures or instructions 1024 (e.g., software) embodying or utilized by any one or more of the techniques or functions described herein. The instructions 1024 may also reside, completely or at least partially, within any of registers of the processor 1002, the main memory 1004, the static memory 1006, or the mass storage 1008 during execution thereof by the machine 1000. In an example, one or any combination of the hardware processor 1002, the main memory 1004, the static memory 1006, or the mass storage 1008 may constitute the machine-readable medium 1022. While the machine-readable medium 1022 is illustrated as a single medium, the term “machine-readable medium” may include a single medium or multiple media (e.g., a centralized or distributed database, and/or associated caches and servers) configured to store the one or more instructions 1024.

The term “machine-readable medium” may include any medium that is capable of storing, encoding, or carrying instructions for execution by the machine 1000 and that cause the machine 1000 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1024 may be further transmitted or received over a communications network 1026 using a transmission medium via the network interface device 1020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.12 family of standards known as Wi-Fi®, IEEE 802.16 family of standards known as WiMax®), IEEE 802.15.4 family of standards, peer-to-peer (P2P) networks, among others. In an example, the network interface device 1020 may include one or more physical jacks (e.g., Ethernet, coaxial, or phone jacks) or one or more antennas to connect to the communications network 1026. In an example, the network interface device 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1200, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system, comprising:
a signal receiver circuit configured to receive physiologic information cyclic with patient respiration and vibration information indicative of patient respiratory vibrations for a plurality of respiratory cycles of a patient; and
an assessment circuit configured to:
identify a first set of respiratory cycles of the plurality of respiratory cycles having a duration within a threshold;
align segments of the vibration information corresponding to the first set of respiratory cycles, the segments associated with a desired portion of the respiratory cycle using a feature of the respiratory cycle; and
determine a composite respiratory vibration using the aligned segments.

2. The system of claim 1, comprising:
an implantable housing comprising a vibration sensor configured to sense the vibration information indicative of patient respiratory vibrations.

3. The system of claim 1, wherein the assessment circuit is configured to determine the composite respiratory vibration to improve a signal-to-noise ratio (SNR) of the vibration information.

4. The system of claim 1, wherein the physiologic information and the vibration information comprise different types of information,
wherein the physiologic information comprises at least of electrocardiogram information of the patient, accelerometer information of the patient, impedance information of the patient, acoustic information of the patient, or blood flow information of the patient.

5. The system of claim 1, wherein the assessment circuit is configured to determine a change in patient status, to detect a physiological condition of the patient, or to determine a patient therapy parameter using the determined composite respiratory vibration.

6. The system of claim 1, wherein the first set of respiratory cycles of the patient comprises at least 3 respiratory cycles.

7. The system of claim 1, wherein the threshold comprises a range within N % of the duration a first respiratory cycle or of the inspiration/expiration (I/E) ratio of the first respiratory cycle.

8. The system of claim 1, wherein the feature of the respiratory cycle comprises a transition between inspiration and expiration of the patient.

9. The system of claim 1, wherein the composite respiratory vibration comprises an average of the aligned vibration information.

10. The system of claim 1, wherein the segments of the vibration information associated with the desired portion of the respiratory cycle comprise segments associated with two or more of:
a wheeze segment of the respiratory cycle;
a stridor segment of the respiratory cycle;
a squawk segment of the respiratory cycle;
a rhonchus segment of the respiratory cycle;
a snore segment of the respiratory cycle;
a fine crackle segment of the respiratory cycle;
a course crackle segment of the respiratory cycle;
a crackle segment of the respiratory cycle; and
a pleural friction rub segment of the respiratory cycle, and
wherein the assessment circuit is configured to determine composite respiratory vibrations for each of the two or more segments.

11. The system of claim 10, wherein the assessment circuit is configured to:
determine trends of the determined composite respiratory vibrations for each of the two or more segments; and
determine a change in patient condition using the determined trends, wherein the change in patient condition includes an indication of at least one of:
chronic obstructive pulmonary disorder (COPD);
asthma;
heart failure (HF);
pneumonia;
bronchitis; or
sleep apnea of the patient.

12. A method, comprising:
receiving, using a signal receiver circuit, physiologic information cyclic with patient respiration for a plurality of respiratory cycles of a patient;
receiving, using the signal receiver circuit, vibration information indicative of patient respiratory vibrations for the plurality of respiratory cycles of the patient;
identifying, using an assessment circuit, a first set of respiratory cycles of the plurality of respiratory cycles having a duration within a threshold;
aligning, using the assessment circuit, segments of the vibration information corresponding to the first set of respiratory cycles, the segments associated with a desired portion of the respiratory cycle using a feature of the respiratory cycle; and
determining, using the assessment circuit, a composite respiratory vibration using the aligned segments.

13. The method of claim 12, comprising:
sensing the vibration information indicative of patient respiratory vibrations using a vibration sensor contained in an implantable housing; and
storing the determined composite respiratory vibration in a memory in the implantable housing,
wherein determining the composite respiratory vibration comprises to improve a signal-to-noise ratio (SNR) of the vibration information.

14. The method of claim 12, wherein the physiologic information and the vibration information comprise different types of information,
wherein the physiologic information comprises at least of electrocardiogram information of the patient, accelerometer information of the patient, impedance information of the patient, acoustic information of the patient, or blood flow information of the patient.

15. The method of claim 12, comprising:
determining a change in patient status, detecting a physiological condition of the patient, or determining a patient therapy parameter using the determined composite respiratory vibration.

16. The method of claim 12, wherein the first set of respiratory cycles of the patient comprises at least 3 respiratory cycles, and
wherein the threshold comprises a range within N % of the duration a first respiratory cycle or of the inspiration/expiration (I/E) ratio of the first respiratory cycle.

17. The method of claim 12, wherein the feature of the respiratory cycle comprises a transition between inspiration and expiration of the patient.

18. The method of claim 12, wherein the composite respiratory vibration comprises an average of the aligned vibration information.

19. The method of claim 12, wherein the segments of the vibration information associated with the desired portion of the respiratory cycle comprise segments associated with two or more of:
a wheeze segment of the respiratory cycle;
a stridor segment of the respiratory cycle;
a squawk segment of the respiratory cycle;
a rhonchus segment of the respiratory cycle;
a snore segment of the respiratory cycle;
a fine crackle segment of the respiratory cycle;
a course crackle segment of the respiratory cycle;
a crackle segment of the respiratory cycle; and
a pleural friction rub segment of the respiratory cycle, and
wherein the assessment circuit is configured to determine composite respiratory vibrations for each of the two or more segments.

20. The method of claim 19, comprising:
determining, using the assessment circuit, trends of the determined composite respiratory vibrations for each of the two or more segments; and
determining, using the assessment circuit, a change in patient condition using the determined trends,
wherein the change in patient condition includes a change in an indication of at least one of:
chronic obstructive pulmonary disorder (COPD);
asthma;
heart failure (HF);
pneumonia;
bronchitis; or
sleep apnea of the patient.
